(19) United States
(12) Patent Application Publication
(10) Pub. No.: US 2023/0027687 A1
(43) Pub. Date: Jan. 26, 2023

(54) LAPAROSCOPIC SURGERY SYSTEM CALIBRATOR AND METHOD FOR USING THE SAME

(71) Applicant: MARINER ENDOSURGERY INC., Hamilton (CA)

(72) Inventors: Bojan NOKOVIC, Etobicoke (CA); Ned NEDIALKOV, Dundas (CA); Mitchell Thomas WILSON, Hamilton (CA)

(21) Appl. No.: 17/758,176

(22) PCT Filed: Dec. 31, 2020
(86) PCT No.: PCT/CA2020/051810

§ 371 Date: Jun. 29, 2022

Related U.S. Application Data
(60) Provisional application No. 62/955,572, filed on Dec. 31, 2019.

(51) Int. Cl. A61B 34/20; A61B 1/313

(52) U.S. Cl. CPC A61B 34/20; A61B 1/3132; A61B 2017/00725

(57) ABSTRACT

A laparoscopic surgery system calibrator and method for using the same are provided. The laparoscopic surgery system calibrator comprises a tool-retaining apparatus and at least one machine. The tool-retaining apparatus is constructed to releasably secure a surgical instrument having at least one fiducial marker thereon. The at least one machine is coupled to the tool-retaining apparatus to pivot the tool-retaining apparatus through a set of poses once the surgical instrument is secured by the tool-retaining apparatus.

### CROSS-REFERENCE TO RELATED APPLICATIONS

This application claims the benefit of U.S. provisional patent application No. 62/955,572, filed Dec. 31, 2019, the contents of which are incorporated herein by reference in their entirety.

### FIELD

The specification relates generally to medical devices. In particular, the following relates to a laparoscopic surgery system calibrator and a method for using the same.

### BACKGROUND OF THE DISCLOSURE

Laparoscopic surgery, also referred to as keyhole surgery, is a relatively-new surgical technique in which operations are performed far from their location through small incisions (usually 0.5-1.5 cm) elsewhere in the body. There are a number of advantages to a patient with laparoscopic surgery versus the more common, open procedure. Pain and hemorrhaging are reduced due to smaller incisions and recovery times are shorter. These reductions are achieved, however, only if the procedure is performed completely and without effective errors. Unfortunately, such errors are not uncommon in laparoscopic surgeries. Indeed, intra-operative and post-operative complications are prevalent with laparoscopic surgery procedures. Because of this, there is a need to improve patient safety during laparoscopic surgery so that the benefits derived from such procedures are achieved while the drawbacks are reduced or eliminated.

One of the most profound drawbacks of laparoscopic surgery is the occurrence of unintentional or inadvertent injuries to patient tissue structures adjacent to or sometimes, distant from the intended surgical site or field. In the pelvic cavity, for example, bowels, ureters, large organs and blood vessels can be injured either directly from the heat or sharpness of the laparoscopic instruments, or burned indirectly through the conduction of heat through nearby tissues. Typically, such injuries are not appreciated at the time of surgery because the specific injury sites are hidden by blood or other patient tissues. As another disadvantage attendant to such iatrogenic injuries, the response to the unintended injury manifested by the patient is often a delayed one. This delayed response can be traumatic as well as tragic, and can sometimes result in one or more further surgeries, which would otherwise be unnecessary.

Reference is made to FIG. 1 which shows a laparoscopic surgery system 20 for use on a body of a patient 24. The laparoscopic surgery system 20 includes a laparoscope 28, a surgical instrument 32, a display 36, a controller 40, and a tracking system 44, which in the illustrated scenario is a camera system. The laparoscope 28 is inserted into the patient 24 via a first incision 48, and has an imaging tip 52 for capturing images of a surgical objective 56 inside the patient 24. The image receiving element may be a lens, for example. During use, the imaging tip 52 is positioned in the body of the patient 24 to receive images of the surgical objective 56. The laparoscope 28 is configured by any suitable means to transmit received images to the controller 40 and/or the display 36. For example, the laparoscope 28 may include an imaging element, such as a lens, and an image sensor (both not shown), which may be, for example, a CCD sensor or a CMOS sensor, that is positioned to receive images from the image receiving element. The laparoscope 28 is configured to transmit the images of the surgical objective 56 to the display 36 (optionally via a controller such as the controller 40). After insertion of the laparoscope 28, the surgical instrument 32 is inserted into the patient 24 via a second incision 60 and is maneuvered towards the surgical objective 56 within the patient 24.

The laparoscopic surgery system 20 is used to determine one or more unsafe zones within the patient 24 through or near which the tip of the surgical instrument 32 should not maneuver to thereby avoid causing injury to the patient 24. The determination of the unsafe zones is performed using images from the laparoscope 28, other imaging means, and general anatomical knowledge, and previous surgical experience.

The tracking system 44 tracks the patient 24 and the surgical instrument 32 during a laparoscopic surgery in order to determine where the surgical instrument 32 is relative to the patient 24 and, in particular, the safe zones. The tracking system 44 includes one or more cameras 64 that are strategically located in an operating theater to view the patient 24, and an external portion 68 of the laparoscope 28 and an external portion 72 of the surgical instrument 32 that extend outside of the body of the patient 24.

By determining the orientation and position of the external portions 68, 72 of the laparoscope 28 and the surgical instrument 32, respectively, and using knowledge of the dimensions of the laparoscope 28 and the surgical instrument 32, the laparoscopic surgery system 20 can model the location of an internal portion 76 of the laparoscope 28 and an internal portion 80 of the surgical instrument 32 to determine their location relative to various physiological regions of the patient 24.

In order to facilitate optical recognition and segmentation of the external portion 72 of the surgical instrument 32 as continuously captured via the tracking system 44 during a surgical procedure, it is known to use fiducial markers on the surgical instrument 32 to determine its orientation and position. The fiducial markers can be any objects that promote visibility to the tracking system 44. Typically, a set of fiducial markers are secured to the surgical instrument 32, often in a known pattern, but in alternative scenarios, the fiducial markers can form part of the surgical instrument. In some scenarios, fiducial markers can have distinctive shapes, such as spheres, stars, polygons, etc. Alternatively and/or additionally, fiducial markers can provide active and/or passive illumination. For example, in some scenarios may include active light elements, such as, for example, light emitting diodes (“LEDs”). In other scenarios, the fiducial markers can provide passive illumination, such as via reflective or retro-reflective surfaces. Common fiducial markers include passive retro-reflective spheres that are used in conjunction with a light source proximate the cameras emitting a light spectrum that is not visible to the human eye so that operating room staff are not distracted. The tracking system 44 is configured to register the infrared light spectrum as it reflects back from the passive retro-reflective fiducial markers in order to recognize their locations. Further, different types of fiducial markers (e.g., shapes, illumination, etc.) may be employed together to facilitate orientation determination. The cameras 64 of the tracking system 44 are intelligent, in that they process registered imaging data to determine the pose of the surgical instrument 32. In particular, the cameras 64 used are Polaris® models from Northern Digital Inc. that emit and image infrared light. Alternatively, a separate computing device can process the imaging data registered by the cameras 64, and this may be performed by the controller 40.

FIG. 2 shows the exemplary surgical instrument 32 for use in laparoscopic surgery with a cluster 82 of fiducial markers 84 secured thereto via a support arm 85. The illustrated exemplary surgical instrument 32 is a pair of laparoscopic scissors, but can be any one of a number of surgical instruments employed in laparoscopic surgery or endoscopic surgery. The surgical instrument 32 has a control end 88 that includes a pair of handles, an operative end 90 that includes a pair of blades, an instrument tip 92 at the end of the operative end 90, and a shaft portion 96 that houses one or more connectors for controlling the blades via the handles. The shaft portion 96 is generally straight and free of bends. The operative end 90 and the shaft portion 96 are configured to be at least partially inserted into the body of the patient 24 through apertures, such as the incision 60. These portions of the surgical instrument 32 are therefore made from materials that will not cause harm to the patient, such as, for example, a suitable stainless steel.

The cluster 82 is designed so that the fiducial markers 84 affixed to it are spatially separated to facilitate their individual recognition and the recognition of the position and orientation of the cluster 82 by the tracking system 44. The fiducial markers 84 have a known spatial relationship in the cluster 82, thus enabling preservation of this known spatial relationship when the cluster 82 is secured to the surgical instrument 32. Prior to surgery, the cluster 82 is secured to the shaft portion 96 of the surgical instrument 32 proximal the control end 88 by a technician, and, understandably, actual placement of the cluster 82 can vary each time.

As the cluster 82 of the fiducial markers 84 is used by the laparoscopic surgery system 20 to determine the location of the instrument tip 92 of the surgical instrument 32, it is desirable to determine the position of the cluster 82 of fiducial markers 84 relative to the instrument tip 92 of the surgical instrument 32 in as precise a manner as possible. As the placement of the cluster 82, and thus the fiducial markers 88 in the cluster 82, varies, the laparoscopic surgery system 20 is calibrated to learn the position of the cluster 82 of fiducial markers 84 relative to the instrument tip 92 after the cluster 82 is secured to the surgical instrument 32. The cluster 82 is generally secured to the surgical instrument 32 prior to each surgery.

FIG. 3 shows a prior art system 100 for calibrating a laparoscopic surgery system 20 for a particular surgical instrument 32 after the cluster 82 of fiducial markers 84 has been affixed to it. The prior art system 100 includes a vessel 104 having a conical divot 108 that has a bottom 112 that is dimensioned for the particular surgical instrument 32. In order to calibrate a laparoscopic surgery system 20 for the surgical instrument 32, the vessel 104 having the appropriately-shaped bottom 112 corresponding to the surgical tool 32 is selected, and the surgical instrument 32 is placed instrument tip 92 first into the conical divot 108. The tracking system 44 is oriented to capture images of the cluster 82 of fiducial markers 84 secured to the surgical instrument 32 in a set of poses (i.e., locations and orientations) as the surgical instrument 32 is manually pivoted around the bottom 112 of the conical divot 108 with the shaft portion 96 sliding along a sidewall 116 of the conical divot 108. During the process of moving the surgical instrument, care must be taken to ensure that the instrument tip 92 does not move from the bottom 112 of the conical divot 108.

The tracking system 44 then processes the registered reflected infrared light to determine the distance between the instrument tip 92 of the surgical instrument 32 and the cluster 82 of fiducial markers 84 using the interpolation to estimate the pivot point and, thus, the location of the instrument tip 92, and subsequently reports the pose of the surgical instrument to the controller 40. This process is, however, quite manual and time-consuming, and can be prone to inconsistent results and errors. Further, a number of vessels are needed as different surgical instruments may require differently dimensioned bottoms.

### SUMMARY OF THE DISCLOSURE

In one aspect, there is provided a laparoscopic surgery system calibrator, comprising a tool-retaining apparatus constructed to releasably secure a surgical instrument having at least one fiducial marker thereon, and at least one machine coupled to the tool-retaining apparatus to pivot the tool-retaining apparatus through a set of poses once the surgical instrument is secured by the tool-retaining apparatus.

The at least one machine can comprise at least one motor.

The tool-retaining apparatus can comprise an at least partial spherical surface, and the at least one machine can engage the at least partial spherical surface to pivot the tool-retaining apparatus and the secured surgical instrument through two degrees of freedom. The at least partial spherical surface can define a pivot point around which the tool-retaining apparatus is pivoted.

The set of poses can be pre-defined.

The tool-retaining apparatus can comprise at least one releasable clamp. The surgical instrument can comprise a generally straight shaft portion, and the at least one clamp can comprise a chuck dimensioned to grasp the generally straight shaft portion of the surgical instrument. The tool-retaining apparatus can comprise a reference surface restricting positioning of the surgical instrument along a longitudinal axis of the generally straight shaft portion when the surgical instrument is aligned for grasping by the chuck. The laparoscopic surgery system calibrator can further comprise a tool cap that is releasably securable to an operative end of the surgical instrument and restricting positioning of the surgical instrument along the longitudinal axis of the generally straight shaft portion by an offset from the reference surface. The first offset can be pre-determined. The surgical instrument can be a first surgical instrument, and the tool cap can be dimensioned to be releasably securable atop of a second surgical instrument that differs in type from the first surgical instrument. The longitudinal axis can be a first longitudinal axis, the pre-determined offset can be a first pre-determined offset, and the tool cap can restrict positioning of the second surgical instrument from the reference surface along a second longitudinal axis of a second shaft portion of the second surgical instrument by a second pre-determined offset. The second pre-determined offset can differ from the first pre-determined offset.

In another aspect, there is provided a laparoscopic surgery system calibrator, comprising a tool-retaining apparatus for releasably securing a surgical instrument, the surgical instrument having at least one fiducial marker thereon, the tool-retaining apparatus comprising an at least partial spherical surface defining a pivot point, and an apparatus support dimensioned to support the tool-retaining apparatus via the at least partial spherical surface to enable pivoting of the tool-retaining apparatus and the secured surgical instrument through two degrees of freedom while generally maintaining the pivot point at a fixed position.

The laparoscopic surgery system calibrator can further comprise at least one motor coupled to the tool-retaining apparatus to pivot the tool-retaining apparatus once the surgical instrument is secured by the tool-retaining apparatus. The at least one motor can pivot the tool-retaining apparatus and the secured surgical instrument through a set of pre-defined poses.

The tool-retaining apparatus can comprise at least one clamp. The surgical instrument can comprise a generally straight shaft portion, and the at least one clamp can comprise a chuck dimensioned to grasp the generally straight shaft portion of the surgical instrument. The tool-retaining apparatus can comprise a reference surface restricting positioning of the surgical instrument along a longitudinal axis of the generally straight shaft portion when the surgical instrument is aligned for grasping by the chuck. The laparoscopic surgery system calibrator can further comprise a tool cap that is releasably securable to an operative end of the surgical instrument restricting positioning of the surgical instrument along the longitudinal axis of the first generally straight shaft portion, and the tool-retaining apparatus can comprise a reference surface restricting positioning of the surgical instrument and the tool cap along the longitudinal axis of the generally straight shaft portion by an offset from the reference surface. The offset can be pre-determined. The surgical instrument can be a first surgical instrument, and the tool cap can be dimensioned to be releasably securable atop of a second surgical instrument that differs in type from the first surgical instrument. The longitudinal axis can be a first longitudinal axis, wherein the shaft portion can be a first shaft portion, wherein the pre-determined offset can be a first pre-determined offset, and the tool cap can restrict positioning of the second surgical instrument from the reference surface along a second longitudinal axis of a second shaft portion of the second surgical instrument by a second pre-determined offset. The second pre-determined offset can differ from the first pre-determined offset.

### BRIEF DESCRIPTIONS OF THE DRAWINGS

For a better understanding of the various embodiments described herein and to show more clearly how they may be carried into effect, reference will now be made, by way of example only, to the accompanying drawings in which:

FIG. 1 shows a laparoscopic surgery system and a patient being operated on;

FIG. 2 shows a surgical instrument that is equipped with a cluster of passive retro-reflective fiducial markers;

FIG. 3 shows a prior art system for calibrating a laparoscopic surgery system for the surgical instrument of FIG. 2;

FIG. 4 is a side view of a laparoscopic surgery system calibrator in accordance with an embodiment;

FIG. 5 is a sectional view of the laparoscopic surgery system calibrator of FIG. 4;

FIG. 6 is a top section view of a tool-retaining apparatus of the laparoscopic surgery system calibrator of FIG. 5;

FIG. 7 is a perspective view of the laparoscopic surgery system calibrator of FIG. 4 showing the degrees of freedom through which a surgical instrument secured therein can be pivoted;

FIG. 8A shows the surgical instrument of FIG. 2 after being fitted with a tool cap;

FIG. 8B is a partial sectional view of the end of the surgical instrument proximate the instrument tip after fitting of the tool cap;

FIG. 9 is a partial sectional view of the surgical instrument and tool cap of FIG. 8A being inserted into the laparoscopic surgery system calibrator of FIG. 4;

FIG. 10A is a partial sectional view of the surgical instrument and tool cap of FIG. 8A after insertion into and securing by the laparoscopic surgery system calibrator of FIG. 4;

FIG. 10B is a partial sectional view of the surgical instrument and the laparoscopic surgery system calibrator of FIG. 10A after pivoting of the tool-retaining apparatus and the secured surgical instrument; and

FIG. 11 shows the calculation of the position of the tip of the surgical instrument relative to the origin of the cluster of fiducial markers.

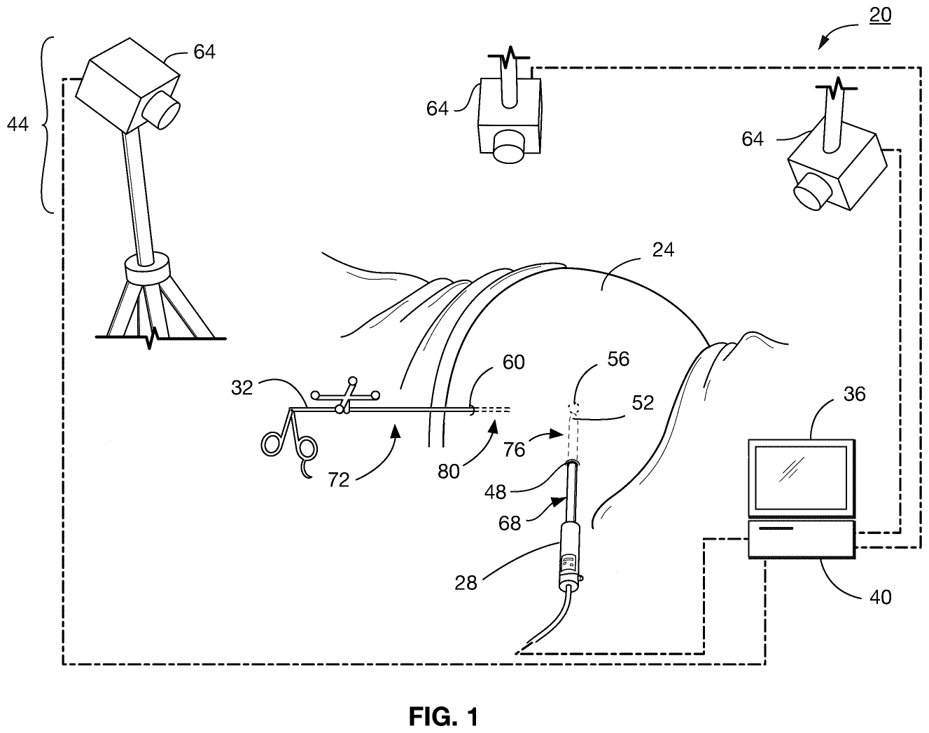

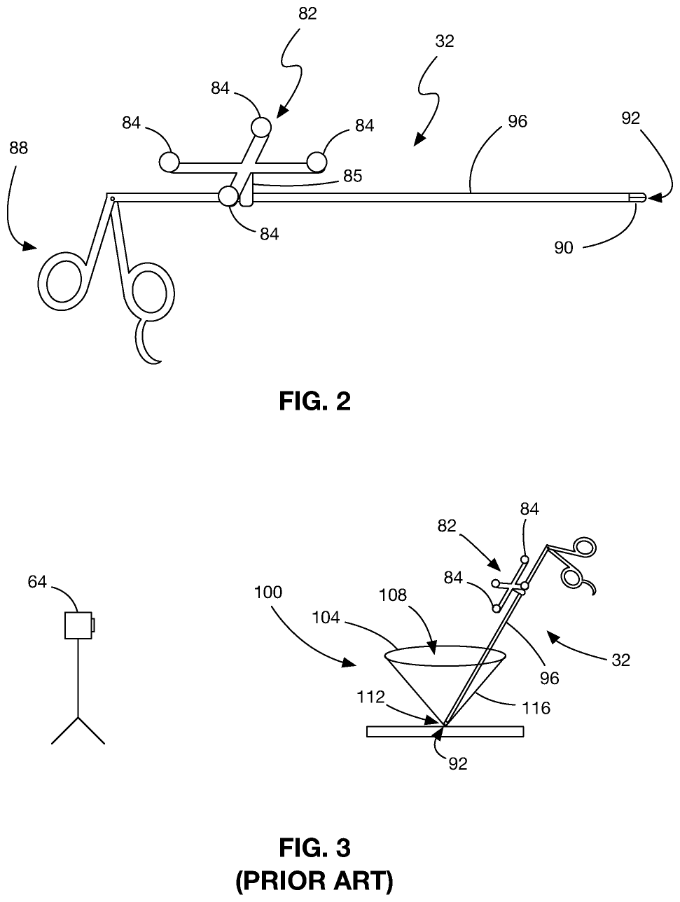

### DETAILED DESCRIPTION

For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

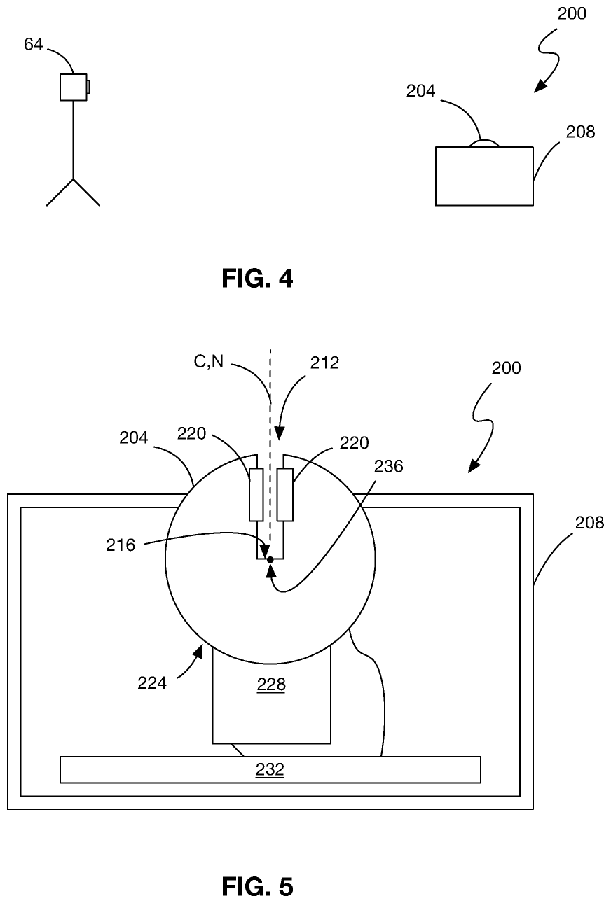

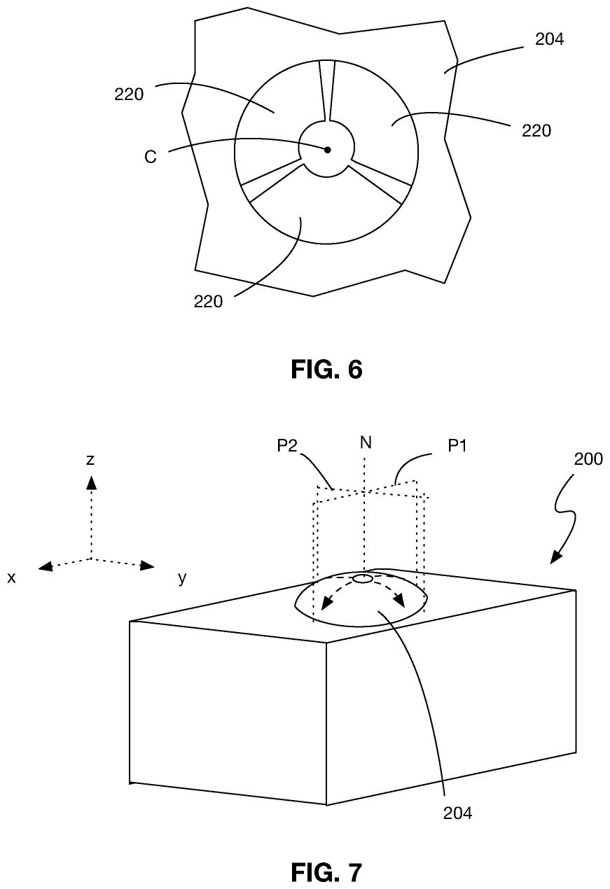

FIGS. 4 to 7 show a laparoscopic surgery system calibrator 200 in accordance with an embodiment. The laparoscopic surgery system calibrator 200 has a tool-retaining apparatus 204 that is generally spherical and housed within a housing 208. The tool-retaining apparatus 204 has a receptacle 212 that is dimensioned to receive the operative end 90 and part of the shaft portion 96 of the surgical instrument 32. The receptacle 212 is a generally circular bore having a reference surface 216 at its lower end. A set of chuck jaws 220 extend into the receptacle 212, as is particularly illustrated in the top view of the receptacle 212 shown in FIG. 6. The chuck jaws 220 are mechanically activated to clamp and unclamp the shaft portions 96 of the surgical instrument 32 inserted into the receptacle 212 to secure the surgical instrument 32 to the tool-retaining apparatus 204. The chuck jaws 220 define a central axis C that is coaxial to the receptacle 212. When the tool-retaining apparatus 204 is in a neutral orientation, the central axis C aligns with a neutral axis N extending vertically from the center of the tool-retaining apparatus 204.

The tool-retaining apparatus 204 has a spherical surface 224 at least on a lower portion thereof that is engaged by a set of servo motors 228. The servo motors 228 act to pivot the tool-retaining apparatus 204 through two degrees of freedom via the spherical surface 224 of the tool-retaining apparatus 204. In particular, the servo motors 228 are arranged in such a way that one of the servo motors 228 pivots the tool-retaining apparatus 204 via the spherical surface 224 such that the surgical instrument 32 is pivoted towards and away from a camera 64, and the other servo motor 228 pivots the tool-retaining apparatus 204 via the spherical surface 224 such that the surgical instrument 32 is pivoted laterally left and right relative to the camera 64 through a plane that is generally normal to the line of sight from the camera 64. While described with respect to servo motors, other types of suitable motors can be employed. A circuit board 232 is coupled to the set of servo motors 228 to control their operation, and to the tool-retaining apparatus 204 to control operation of the chuck jaws 220. In the illustrated embodiment, the circuit board 232 is a Performance TM4C123GH6PM MCU. The circuit board 232 includes at least one processor and a memory, and is programmed via any suitable means to control a first of the servo motors 228 to pivot the tool-retaining apparatus 204 within a range between 45 degrees on either side of the neutral axis N along a first plane P1 parallel to the neutral axis N and the x-axis, and to control a second of the servo motors 228 to pivot the tool-retaining apparatus 204 within a range between 45 degrees on either side of the neutral axis N along a second plane P2 parallel to the neutral axis N and the y-axis and perpendicular to the first plane P1. The laparoscopic surgery system calibrator 200 is designed to pivot the surgical instrument 32 through a similar range of motion as is possible using the prior art system shown in FIG. 3. The computer-readable instructions used to program the circuit board 232 can be provided via firmware or software stored in the memory, a system on a chip, an application-specific integrated circuit (“ASIC”), etc. The circuit board 232 can additionally include internal or external storage for storing data for each calibration, and a network module for wired or wireless communications for communicating calibration data to a networked computer. A user-operable control (a physical button in the present embodiment) toggles the clamping and unclamping of the surgical instrument 32 via the chuck jaws 220. The circuit board 232 is in communication with the controller 40, which directs the laparoscopic surgery system calibrator 200 to commence a calibration.

The spherical surface of the tool-retaining apparatus 204 defines a pivot point 236 around which the tool-retaining apparatus 204 is pivoted when the servo motors 228 rotate the spherical surface 224. The pivot point 236 is generally equidistant from each point on the spherical surface 224. The depth of the reference surface 216 within the receptacle 212 is selected such that the reference surface 216 coincides with the pivot point 236 so that the relationship between the instrument tip 92 of the surgical instrument 32 and the pivot point 236 is known. In other embodiments, the reference surface can be positioned such that it is not co-located with the pivot point, but has a known relationship to the pivot point (e.g., the reference surface is positioned 5 mm past the pivot point within the receptacle).

In the illustrated embodiment, the tool-retaining apparatus 204 is generally spherical, but in other embodiments, it may be desirable for the tool-retaining apparatus 204 to have at least a partial spherical surface along its lower side to facilitate smooth pivoting via machinery such as motors, and can be non-spherical on its upper surface as this portion is not acted on by the machinery to pivot the tool-retaining apparatus.

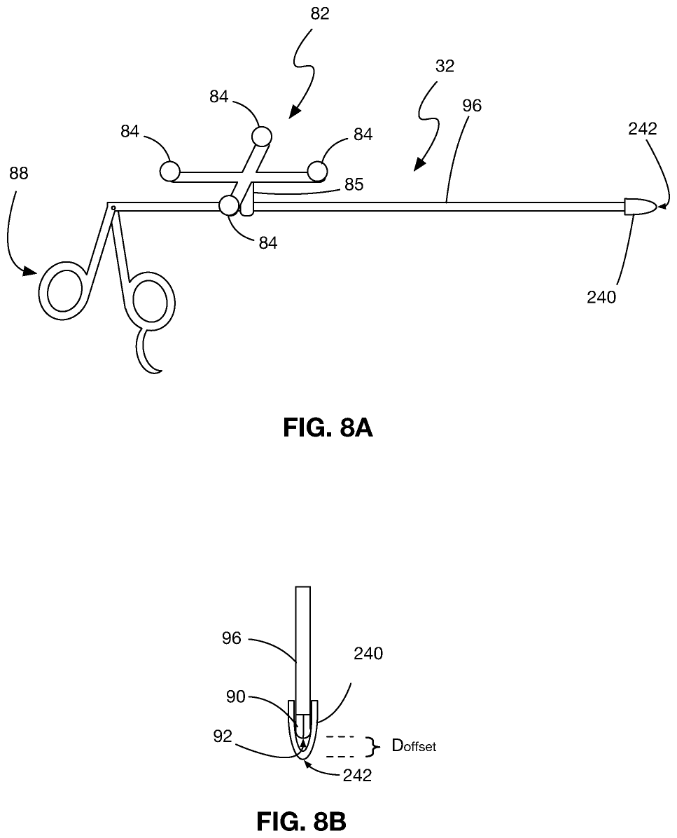

FIG. 8A shows the surgical instrument 32 after deployment of a tool cap 240. The tool cap 240 covers the instrument tip 92 of the surgical instrument 32, and maintains the sterility of the surgical instrument 32 during the calibration process. The tool cap 240 is readily sterilized between calibrations, or, alternatively, can be made to be disposable after a calibration. The instrument tip 92 of the surgical instrument 32 is covered by the tool cap 240 in a predictable manner, in that the offset of the instrument tip 92 from a tip 242 of the tool cap 240 is generally known.

FIG. 8B shows the offset D_{offset} of the instrument tip 92 from the tip 242 of the tool cap 240 after placement of the tool cap 240 over the instrument tip 92. The offset D_{offset} is pre-determined for each type of surgical instrument, and stored in a table in a memory of the tracking system 44. In other embodiments, the offset D_{offset} can also be stored or indicated elsewhere, such as in the memory of the circuit board 232 and communicated to the tracking system 44, or visually associated with the surgical instrument 32. An identifier of the surgical instrument 32 can be keyed in manually to enable the tracking system 44 to associate the surgical instrument 32 with a particular offset in the presently-described embodiment, but can be alternatively or additionally determined in other ways, such as by recognizing the surgical instrument 32 via its shape and/or markings, or via characteristics of the fiducial markers 84 secured to the surgical instrument 32.

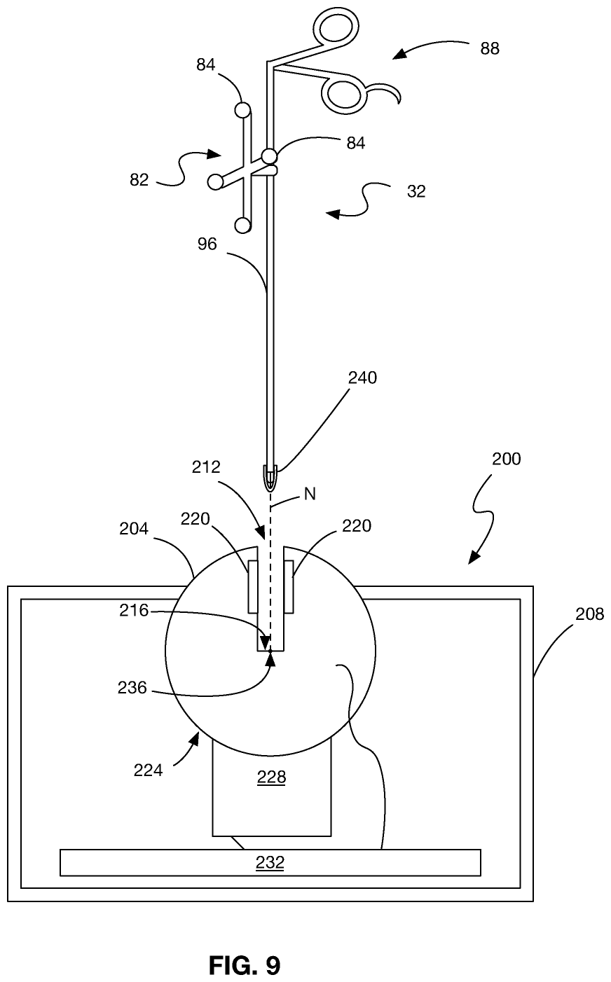

FIG. 9 is a partial sectional view of the surgical instrument 32 just prior to being inserted into the tool-retaining apparatus 204 of the laparoscopic surgery system calibrator 200 after deployment of the tool cap 240 over its operative end 90. Prior to insertion of the surgical instrument 32, a user causes the laparoscopic surgery system calibrator 200 to open the chuck jaws 220 by withdrawing them generally radially from the receptacle 212 to facilitate passage of the surgical instrument 32 and the deployed tool cap 240 towards the reference surface 216. As shown, the shaft portion 96 of the surgical instrument 32 is aligned generally axially, instrument tip 92 down, with the central axis C of the receptacle 212, and rotated about the longitudinal axis of the shaft portion 96 such that the cluster 82 of fiducial markers 84 faces the cameras 64 of the tracking system 44. Once so aligned, the surgical instrument 32 is then inserted into the receptacle 212 by lowering it until the tip 242 of the tool cap 240 abuts the reference surface 216. The shaft portion 96 is generally coaxial with a central axis C of the receptacle 212 that is also coaxial with the neutral axis N in the orientation of the tool-retaining apparatus 204 shown in FIG. 9.

Once the surgical instrument 32 is lowered into the receptacle 212 of the tool-retaining apparatus 204, the user causes the laparoscopic surgery system calibrator 200 to close the chuck jaws 220 by moving them radially towards the central axis C of the receptacle 212. The design of the chuck jaws 220 is such that they grasp the shaft portion 96 of the surgical instrument 32 in a manner that secures the shaft portion 96 so that it is coaxial with the central axis C of the receptacle 212.

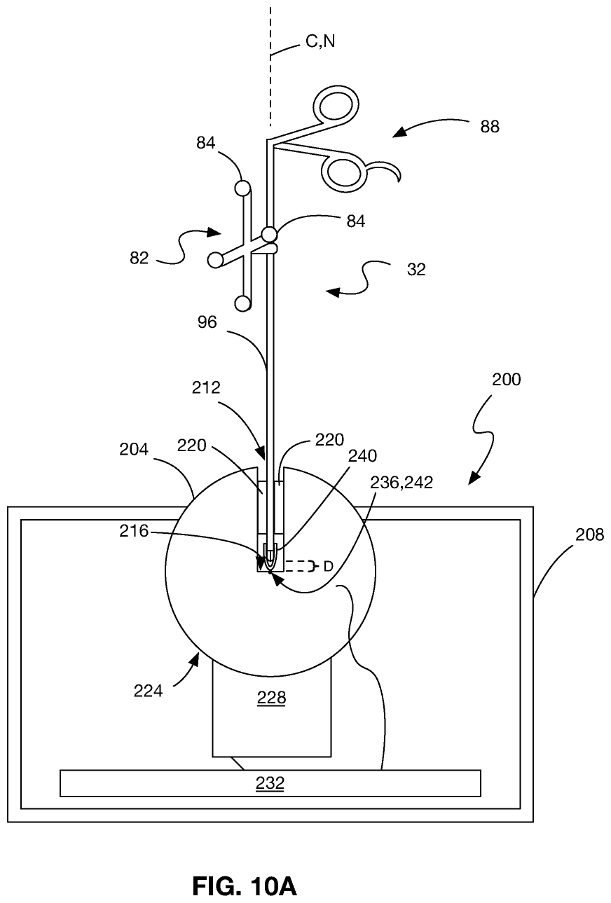

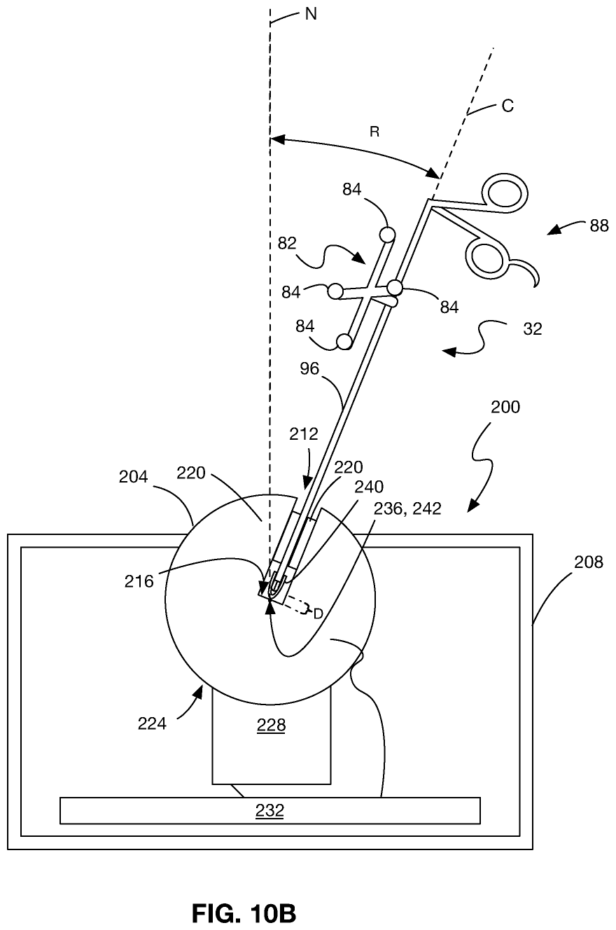

FIG. 10A shows the surgical instrument 32 and the tool cap 240 after insertion into the receptacle 212 of the tool-retaining apparatus 204 and clamping of the chuck jaws 220 on the shaft portion 96 of the surgical instrument 32. As can be seen, the tip 242 of the tool cap 240 abutting against the reference surface 216 is effectively co-located with the pivot point 236 of the tool-retaining apparatus 204. Once the shaft portion 96 of the surgical instrument 32 is clamped by the chuck jaws 220, the tool-retaining apparatus 204 and the surgical instrument 32 can be pivoted about the pivot point 236 and the co-located tip 242 of the tool cap 240 by the servo motors 228.

The cluster 82 of fiducial markers 84 has a unique geometry, and a description and precise location of the fiducial markers 84 in the cluster 82 is well known by the tracking system 44 and verified before use. In addition to this, a local coordinate system with an origin in one of the fiducial markers 84 in the cluster 82 (or geometric center of selected fiducial markers 84) is known to the tracking system 44.

FIG. 10B shows the laparoscopic surgery system calibrator 200 after pivoting of the tool-retaining apparatus 204 and the secured surgical instrument 32 through a rotation of R about the pivot point 236 and the co-located tip 242 of the tool cap 240 to the shown resulting pose during the process of calibration. The central axis C of the receptacle 212 and the shaft portion 96 of the surgical instrument 32 secured therein is no longer coaxial with the neutral axis N. As shown, the tip 242 of the tool cap 240 and the pivot point 236 remain in a fixed location while the tool-retaining apparatus 204 and the secured surgical instrument 32 are pivoted about the pivot point 236, enabling the tracking system 44 to observe and model movement of the cluster 82 of the fiducial markers 84 and determine the relative distance to the pivot point 236 and, thus, the co-located tip 242 of the tool cap 240. Once the distance to the tip 242 of the tool cap 240 has been calculated, it can be adjusted for the offset D_{offset} between the instrument tip 92 and the tip 242 of the tool cap 240.

The calibration process takes approximately 20 seconds, but can be configured to be longer of shorter. During this time, the instrument is moved 30 to 60 degrees forward-backward and left-right from the initial position shown in FIG. 10A. The tracking system 44 registers the position of the fiducial markers 84, and thus the origin, at a rate of 60 frames per second, so that, in the 20 second period during the calibration process, the cameras 64 record about 1200 frames. Based on the observed movement pattern of the origin of the cluster 82 of the fiducial markers 84, the tracking system 44 determines the location of the pivot point 236 and the radius of movement of the origin relative to the pivot point 236 using known techniques. Further, the tracking system 44 uses knowledge of the position of the reference surface 216 relative to the pivot point 236 (in this embodiment, they are co-located), and the offset D_{offset} for the instrument tip 92, in order to determine the location of the instrument tip 92 relative to the origin of the cluster 82 of fiducial markers 84. The distance of the origin of the cluster 82 of the fiducial markers 84 from the axis of the surgical instrument 32 need not be known in advance, but it is directly related to the collected origin positions. The approach used to find the center and radius of the sphere along which the origin moves during the calibration process is based on W. H. Beyer method (http://math.stackexchange.com/tags/centroid/hot).

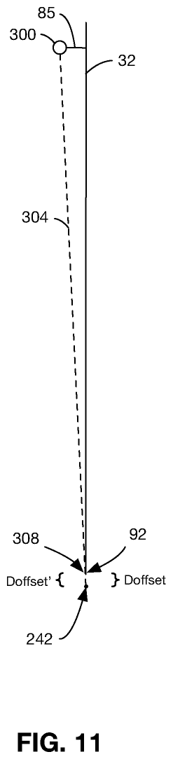

FIG. 11 shows the estimation of the location of the instrument tip 92 of the surgical instrument 32 relative to the cluster of fiducial markers in greater detail. The origin 300 is determined for the cluster of fiducial markers and may or may not coincide with the location of any one of the fiducial markers. In one embodiment, the origin 300 represents a central position between the fiducial markers. The origin 300 is displaced from the surgical instrument 32 by the support arm 85. A radius 304 between the origin 300 and the tip 242 of the tool cap is determined by the cameras 64 during calibration. The offset D_{offset} represents the distance between the tip 242 of the tool cap and the instrument tip 92 along the central axis of the surgical instrument 32. While the radius 304 from the origin 300 to the tip 242 of the tool cap is not exactly co-axial with the central axis of the surgical instrument 32, the angular displacement between the radius 304 and the central axis of the surgical instrument 32 is small enough such that direct adjustment of the radius 300 by the offset D_{offset} (shown as D_{offset}′) results in an estimated instrument tip position 308 that is spaced from the actual instrument tip 92 by a negligible distance. The spatial displacement of the estimated instrument tip position 308 relative to the origin 300 is determined and registered for the surgical instrument 32 as part of the calibration process.

Upon calibrating the laparoscopic surgery system 20 for the surgical instrument 20, the user causes the laparoscopic surgery system calibrator 200 to unclamp the surgical instrument 32 by withdrawing the chuck jaws 220 away from the center axis C of the receptacle 212. The surgical instrument 32 can then be withdrawn from the receptacle 212 and the tool cap 240 can be removed from it so that the surgical instrument 32 is ready to be used with the laparoscopic surgery system 20.

During a surgical procedure, the tracking system 44 registers infrared light reflected from the cluster 82 of the fiducial markers 84 attached to the surgical instrument 32. The number of cameras 64 of the tracking system 44 and their positions are selected to satisfactorily register infrared light from the cluster 82 of the fiducial markers 84 secured to the surgical instrument 32. The tracking system 44 uses the registered reflected infrared light to identify the fiducial markers 84. The relative locations of the fiducial markers 84 are used by the cameras 64 to determine the pose of the cluster 82 (that includes the absolute position of the origin of the cluster 82 and the orientation of the cluster 82). The pose of the cluster 82 and the spatial displacement of the estimated instrument tip position relative to the origin are used to estimate the absolute position of the instrument tip.

The position and orientation of the surgical tool 32 is then reported by the tracking system 44 to the controller 40 as a message that includes the absolute position (x, y, z) of the origin 300 of the cluster 82, the rotation (Rx, Ry, Rz) of the cluster 82, and the estimated absolute position of the instrument tip 92. The controller 40 can then use the pose of the cluster 82 and the estimated position of the instrument tip 92 communicated by the cameras 64 to relate the position of the instrument tip 92 of the surgical instrument 32 to other objects, such as the anatomy of the subject, in order to present virtual images of the surgical procedure and detect when the instrument tip 92 of the surgical instrument 32 is approaching or within an unsafe zone, and provide a warning. The warning can be a visual warning presented on the display 36. Alternatively or additionally, the warning may be an audible warning generated by the controller 40 or another connected device.

It has been determined that, in various testing scenarios, that the reported location of the origin is within less than 0.75 mm from its actual position on a virtual spherical surface having a radius equal to the distance from the origin to the instrument tip 92 of the surgical tool 32.

While, in the above-described embodiment, the tool-retaining apparatus is pivoted via motors, in other embodiments, the tool-retaining apparatus may be pivoted via one or more machines, such as ones actuated manually via a crank or the like.

The tool-retaining apparatus is any apparatus that is constructed to secure and enable pivoting of a laparoscopic or endoscopic surgical instrument in different poses in a pattern that enables the spatial relationship between the fiducial markers and the parts of the surgical instrument, such as the instrument tip, to be determined. The tool-retaining apparatus can include a releasable clamp of some sort, such as a chuck as described above, a bar clamp, a band clamp, a spring clamp, an aperture with a set screw, a quick-release clamp, a magnetic clamp, etc. Additionally or alternatively, the tool-retaining apparatus can include an aperture that is constructed to releasably secure a surgical instrument, such as via a friction fit.

The tool-retaining apparatus can have an at least partial spherical surface that defines a pivot point around which the tool-retaining apparatus can be pivoted when supported by an apparatus support. The tool-retaining apparatus can be pivoted by hand or by a machine, such as via one or more motors. The apparatus support can be any device that supports the tool-retaining apparatus in a manner that enables it to pivot.

The tool-retaining apparatus can be pivoted by at least one machine coupled to it to pivot the tool-retaining apparatus through a set of poses once a surgical instrument is secured by the tool-retaining apparatus. The at least one machine can be manually operated in some embodiments. In other embodiments, the at least one machine can be motors acting to pivot the tool-retaining apparatus, such as by friction torque on an at least partial spherical surface of the tool-retaining apparatus.

In the above-described embodiment, the surgical instrument is pivoted through a set of poses and imaged while moving. In other alternative embodiments, the surgical instrument is pivoted between poses, then held stationarily in the poses while being imaged. The poses can be pre-defined to facilitate orientation discovery by the laparoscopic surgery system.

Other approaches can be employed to secure the surgical instrument to the tool-retaining apparatus. For example, various other types of clamps can be employed. In another embodiment, a tool cap and a receptacle within the tool-retaining apparatus are dimensioned such that the tool cap is snugly held within the receptacle during pivoting of the tool-retaining apparatus.

Different tool caps having different offsets can be employed in other embodiments.

Persons skilled in the art will appreciate that there are yet more alternative implementations and modifications possible, and that the above examples are only illustrations of one or more implementations. The scope, therefore, is only to be limited by the claims appended hereto.

1. A laparoscopic surgery system calibrator, comprising:
a tool-retaining apparatus constructed to releasably secure a surgical instrument having at least one fiducial marker thereon; and
at least one machine coupled to the tool-retaining apparatus to pivot the tool-retaining apparatus through a set of poses once the surgical instrument is secured by the tool-retaining apparatus.

2. A laparoscopic surgery system calibrator according to claim 1, wherein the at least one machine comprises at least one motor.

3. A laparoscopic surgery system calibrator according to claim 1, wherein the tool-retaining apparatus comprises an at least partial spherical surface, and wherein the at least one machine engages the at least partial spherical surface to pivot the tool-retaining apparatus and the secured surgical instrument through two degrees of freedom.

4. A laparoscopic surgery system calibrator according to claim 3, wherein the at least partial spherical surface defines a pivot point around which the tool-retaining apparatus is pivoted.

5. A laparoscopic surgery system calibrator according to claim 1, wherein the set of poses are pre-defined.

6. A laparoscopic surgery system calibrator according to claim 1, wherein the tool-retaining apparatus comprises at least one releasable clamp.

7. A laparoscopic surgery system calibrator according to claim 6, wherein the surgical instrument comprises a generally straight shaft portion, and the at least one clamp comprises a chuck dimensioned to grasp the generally straight shaft portion of the surgical instrument.

8. A laparoscopic surgery system calibrator according to claim 7, wherein the tool-retaining apparatus comprises a reference surface restricting positioning of the surgical instrument along a longitudinal axis of the generally straight shaft portion when the surgical instrument is aligned for grasping by the chuck.

9. A laparoscopic surgery system calibrator according to claim 8, further comprising a tool cap that is releasably securable to an operative end of the surgical instrument and restricting positioning of the surgical instrument along the longitudinal axis of the generally straight shaft portion by an offset from the reference surface.

10. A laparoscopic surgery system calibrator according to claim 9, wherein the offset is pre-determined.

11. A laparoscopic surgery system calibrator according to claim 10, wherein the surgical instrument is a first surgical instrument, and wherein the tool cap is dimensioned to be releasably securable atop of a second surgical instrument that differs in type from the first surgical instrument.

12. A laparoscopic surgery system calibrator according to claim 11, wherein the longitudinal axis is a first longitudinal axis, wherein the pre-determined offset is a first pre-determined offset, and wherein the tool cap restricts positioning of the second surgical instrument from the reference surface along a second longitudinal axis of a second shaft portion of the second surgical instrument by a second pre-determined offset.

13. A laparoscopic surgery system calibrator according to claim 12, wherein the second pre-determined offset differs from the first pre-determined offset.

14. A laparoscopic surgery system calibrator, comprising:
a tool-retaining apparatus for releasably securing a surgical instrument, the surgical instrument having at least one fiducial marker thereon, the tool-retaining apparatus comprising an at least partial spherical surface defining a pivot point; and
an apparatus support dimensioned to support the tool-retaining apparatus via the at least partial spherical surface to enable pivoting of the tool-retaining apparatus and the secured surgical instrument through two degrees of freedom while generally maintaining the pivot point at a fixed position.

15. A laparoscopic surgery system calibrator of claim 14, further comprising:
at least one motor coupled to the tool-retaining apparatus to pivot the tool-retaining apparatus once the surgical instrument is secured by the tool-retaining apparatus.

16. A laparoscopic surgery system calibrator according to claim 15, wherein the at least one motor pivots the tool-retaining apparatus and the secured surgical instrument through a set of pre-defined poses.

17. A laparoscopic surgery system calibrator according to claim 14, wherein the tool-retaining apparatus comprises at least one clamp.

18. A laparoscopic surgery system calibrator according to claim 17, wherein the surgical instrument comprises a generally straight shaft portion, and the at least one clamp comprises a chuck dimensioned to grasp the generally straight shaft portion of the surgical instrument.

19. A laparoscopic surgery system calibrator according to claim 18, wherein the tool-retaining apparatus comprises a reference surface restricting positioning of the surgical instrument along a longitudinal axis of the generally straight shaft portion when the surgical instrument is aligned for grasping by the chuck.

20. A laparoscopic surgery system calibrator according to claim 19, further comprising a tool cap that is releasably securable to an operative end of the surgical instrument and restricting positioning of the surgical instrument along the longitudinal axis of the generally straight shaft portion, and wherein the tool-retaining apparatus comprises a reference surface restricting positioning of the surgical instrument and the tool cap along the longitudinal axis of the generally straight shaft portion by an offset from the reference surface.

21.-24. (canceled)
